(19) United States
(12) Patent Application Publication
(10) Pub. No.: US 2023/0200833 A1
(43) Pub. Date: Jun. 29, 2023

(54) SURGICAL CUTTING INSTRUMENT

(71) Applicants: Paul D. PATERSON, West Falls, NY (US); Michele PRESSACCO, Martignacco (IT); Christoph FIEDLER, Laage (DE)

(72) Inventors: Paul D. PATERSON, West Falls, NY (US); Michele PRESSACCO, Martignacco (IT); Christoph FIEDLER, Laage (DE)

(21) Appl. No.: 18/083,849

(22) Filed: Dec. 19, 2022

Related U.S. Application Data
(60) Provisional application No. 63/294,130, filed on Dec. 28, 2021.

(51) Int. Cl. A61B 17/32

(52) U.S. Cl. CPC A61B 17/320036; A61B 2017/320052

(57) ABSTRACT

The present invention relates to a surgical cutting instrument for minimally invasive surgery comprising a gripping element, a hollow stem mounted as an extension of said gripping element along a longitudinal axis of the instrument; a tie rod housed in the hollow stem and having a proximal end coupled to a control lever in said gripping element and a distal end hinged to a blade element; a covering and guiding sheath for said blade element as an extension of said stem. The blade element has a fulcrum that is movable in said covering and guiding sheath and is configured to pass from an inactive position in which it is hidden housed in said covering sheath and an active position protruding outwardly through a window of said covering sheath and vice versa.

### FIELD OF THE INVENTION

The present invention relates to a surgical cutting instrument for minimally invasive surgery.

In general, the present invention finds application in the surgical field, in particular for the interventions related to carpal tunnel, and even more specifically, but not limitedly, to interventions for the release of the carpal tunnel by cutting the transverse ligament by means of a minimally invasive ultrasound-guided technique.

### PRIOR ART

It is known from statistical data that 3.7% of the general population suffers from disorders related to carpal tunnel syndrome and even 7% of unskilled workers.

In the United States the incidence of carpal tunnel syndrome (CTS) is 12,000,000 people.

500,000 people are treated surgically each year through a procedure known as the carpal tunnel release (CTR).

During the carpal tunnel release procedure the transverse carpal ligament is cut in order to reduce the compression of the median nerve and the pressure in the carpal tunnel region.

50% of carpal tunnel release procedures are made through a so-called “open” surgical technique, the remaining 50% through an endoscopic technique.

Although these surgical techniques report good clinical results in at least 97% of cases, many people prefer not to undergo any treatment in order to avoid a long period of convalescence and a long clinical course which requires several specialist visits within 6-9 months (typically 5-6 visits).

Minimally invasive ultrasound-guided techniques have recently been developed in order to overcome the limitations of conventional techniques and therefore reduce recovery times, reduce pain, avoid stitches, improve patient satisfaction, and therefore reduce treatment costs.

In implementing these techniques, it is important that the cutting blade is covered both when the surgeon inserts the instrument into the carpal canal and when he extracts it, in order to avoid accidental damage to the tissues of this anatomical site.

The carpal canal is a cavity located at the level of the wrist.

In the anatomical position there are the carpal bones posteriorly and the transverse carpal ligament anteriorly.

In addition to the median nerve, the veins and tendons of the flexor muscles of the fingers pass through the tunnel thus formed.

It is therefore well understood that in such a small space it is of fundamental importance to extract the blade only when there is absolute certainty that only the tissue of interest will be cut, i.e. the transverse carpal ligament.

It is also preferable for the blade to be covered when the instrument is not being used.

US 2017/0042565 A1, U.S. Pat. No. 10,357,272 B2, EP 3193748 B1 patent applications present a solution in this regard.

In particular, the cutting element comes out of the seat due to the elastic deformation of the element itself and, even more specifically, the extraction of the blade takes place through defined paths thanks to the elastic deformation of the stem on which it is mounted.

This solution certainly has the advantage of being simple and thus very cheap.

However, there is also the obvious disadvantage of having a cutting instrument that is not particularly rigid.

Vice versa, it would be difficult to elastically deform the blade with just the push of the fingers of the surgeon who is operating the instrument.

This represents a limitation because on the one hand the deformability of the instrument prevents the surgeon from having a haptic perception in the moment when he operates, on the other hand in the case of tissues, such as for instance particularly tenacious ligaments, it is difficult to make the cut safely.

For this reason, the technical problem underlying the present invention is to provide an instrument able to overcome the above limits of the prior art.

An object of the invention is to make a rigid instrument in order to give the surgeon a haptic perception.

Still an object of the present invention is to provide an instrument able to facilitate the detachment of the soft tissues of the palmar arch, in particular prior to carrying out the cut of the transverse carpal ligament.

A further object is to provide an instrument which allows having reference markers in the instrument that are distinguishable from the images obtained by ultrasound examination.

Finally, an object of the invention is to provide a solution that is easy to use daily for a surgeon.

### SUMMARY OF THE INVENTION

The solution idea underlying the present invention is to provide an instrument which maintains the advantages deriving from a hidden solution, but which does not require an elastic deformation for the extraction and retraction phases.

The above technical problem is solved by a surgical cutting instrument for minimally invasive surgery comprising a gripping element, a hollow stem mounted as an extension of the gripping element along a longitudinal axis of the instrument and a tie rod housed in the hollow stem and having a proximal end coupled to a control lever in the gripping element and a distal end hinged to a blade element.

In other words, the blade is not obtained integrally with the tie rod but assembled with a pin which therefore allows the rotation thereof about an axis.

Moreover the cutting instrument comprises a covering guiding sheath for the blade element as an extension of the stem.

The blade element has a movable fulcrum in the covering and guiding sheath and is configured to pass from an inactive position in which it is hidden housed in the covering sheath and an active position protruding outwardly through a window of the covering sheath and vice versa.

The fulcrum is the material point representing the movement of the blade element, the rotation along an arch of said fulcrum bringing the blade element from an inactive position in which it is hidden housed in the covering sheath and is parallel to the longitudinal development of the cutting instrument to an active position to carry out the cutting and transversal, substantially orthogonal, with respect to the longitudinal development of the cutting instrument.

Advantageously, the present solution allows having an instrument with a hidden blade, adapted to perform the minimally invasive surgery, but having such a rigidity as to allow the surgeon to maintain the perfect haptic perception for the execution of the operation.

Moreover, advantageously, thanks to the presence of the control lever, the surgeon can proceed with one hand and in a simple and intuitive way to pass the cutting instrument from an inactive phase to an active phase and vice versa.

Preferably, the distal end of the tie rod is U-shaped, as an arch ring, and comprises a pair of engage holes facing each other.

The blade element comprises a proximal hole coaxially aligned with the engage holes to receive a pin so as to make a hinging connection between tie rod and blade element.

Advantageously, the present solution it is both simple to implement and very effective to allow the rotation of the blade element with respect to the tie rod.

According to a preferred embodiment of the invention, the blade element comprises a second pin at the fulcrum and the covering sheath comprises at least one inner guide groove.

The second pin is adapted to be movably guided by the tie rod in the at least one inner guide groove making the fulcrum movable.

The second pin is thus adapted to slide along the inner guide rail to allow a movement of the tie rod and the blade element.

Thus, according to said embodiment, the complete extraction and fold-out mechanism of the blade comprises both a joint mechanism at the hinge and a system that drives a pin integral with the blade element.

The second pin may be made integral with the blade element, or it may be connected and made integral thereto.

According to an alternative embodiment of the invention, the blade element comprises at least one race element and the covering sheath comprises at least one guide element.

The at least one race element is adapted to operationally abut with the at least one guide element by a movement of the tie rod, making the fulcrum movable.

In other words, in this case the rotation of the fulcrum is obtained thanks to a specific shape of the blade and at least one guide element in the covering sheath, whose mutual abutment allows the passage of the blade from an active position to an inactive position and vice versa.

Advantageously, the present solution allows reducing the number of components required while still ensuring a correct implementation of the blade element and tie rod movement.

Nothing prevents a combination of the embodiments considered above, since there are no constraints that prevent the creation of embodiments with mixed solutions.

Still preferably, the covering sheath comprises two portions facing each other with a left covering half shell and a right covering half shell coupled to each other to enclose both the end portion of the tie rod and the blade element.

Advantageously, the present solution allows speed and precision both in the production phase and in the assembly phase of the surgical cutting instrument, ensuring perfect covering of the blade element and the correct position thereof both in the active phase and in the inactive phase

Preferably, the surgical cutting instrument further comprises a tip coupled at the blade element and comprising a protuberance for the detachment of the soft tissues. The protuberance is further adapted to provide a spatial positioning feedback of the surgical cutting instrument in the working condition.

Advantageously, the present solution allows detaching and removing the soft tissues prior to carrying out the cutting of the ligament, thus increasing the safety of the intervention and also allows the surgeon to know where the instrument is when inserted.

Furthermore preferably, the control lever is housed and sliding at a seat formed in the gripping element.

Advantageously, this solution is practical for the surgeon and makes the overall cutting instrument compact.

Preferably, the gripping element as well comprises a left gripping half shell and a right gripping half shell coupled to each other so as to enclose a portion of the hollow stem.

Advantageously, this solution, similarly to the cover mechanism, is optimal for the production and assembly phases.

Preferably, the blade element is C-shaped or according to an angle of 135°, thus cutting as a knife.

Advantageously, the present solution is optimal as regards the intervention at the carpal tunnel.

Still preferably, the cutting instrument according to the invention has vertical reference recesses which can be detected by ultrasounds to identify a position of the instrument with respect to the surrounding tissues.

Advantageously, this, generally but not exclusively along with a characteristic shape of the tip, facilitates the surgeon in the correct identification of the position of the instrument with respect to the surrounding tissues.

Further features and advantages will become more apparent from the following detailed description of a preferred, but not exclusive, embodiment of the surgical cutting instrument according to the present invention, with reference to the enclosed figures given by way of non-limiting example.

### BRIEF DESCRIPTION OF THE DRAWINGS

In these drawings:

FIG. 1 shows a perspective view of a cutting instrument according to a first embodiment according to the invention.

FIG. 2 shows an exploded view of the cutting instrument of FIG. 1.

FIG. 3 shows an exploded view of a detail of the cutting instrument of FIG. 1.

FIG. 4 shows a perspective view of a blade element of the cutting instrument of FIG. 1.

FIGS. 5A and 5B show two positions of a blade element with respect to a tie element in the cutting instrument of FIG. 1.

FIG. 6 shows a perspective view of a covering sheath of the cutting instrument of FIG. 1.

FIG. 7 shows a side view of the covering sheath of FIG. 6.

FIG. 8 shows a tip of the cutting instrument of FIG. 1.

FIG. 9 shows an exploded view of another detail of the cutting instrument of FIG. 1.

FIG. 10 shows a sectional view of a sliding lever of the cutting instrument of FIG. 1.

FIG. 11 shows a sectional view of the sliding lever of FIG. 10 with a tie element in the cutting instrument of

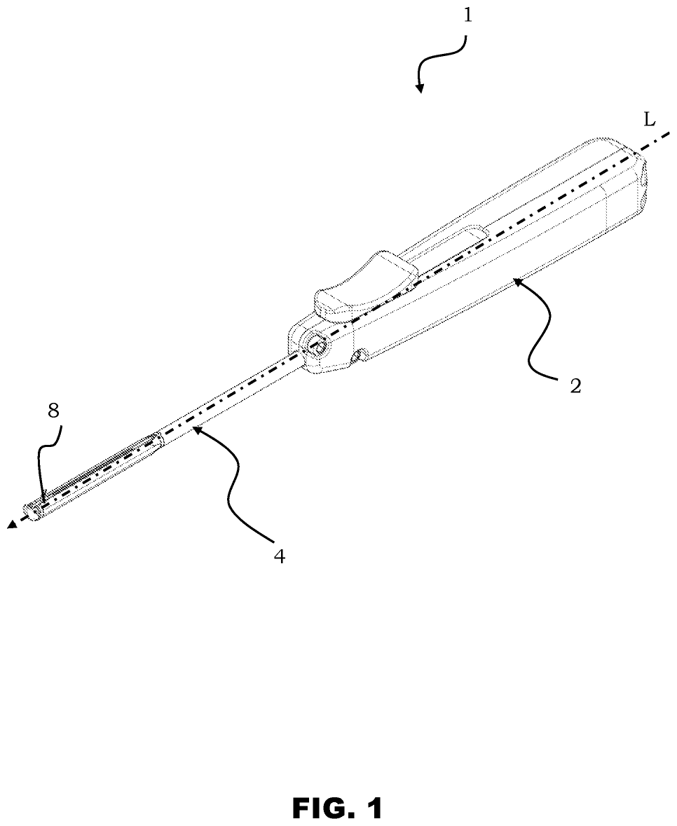

FIG. 1.

FIG. 12 shows a movement simulation of the blade element in the cutting instrument of FIG. 1.

FIG. 13 shows another movement simulation of the blade element in the cutting instrument of FIG. 1.

FIG. 14 shows a perspective view of a cutting instrument according to a second embodiment according to the invention.

FIG. 15 shows a perspective view of a blade element of the cutting instrument of FIG. 14.

FIG. 16 shows a perspective view of a covering sheath of the cutting instrument of FIG. 14.

FIG. 17 shows a movement simulation of the blade element in the cutting instrument of FIG. 14.

FIG. 18 shows another movement simulation of the blade element in the cutting instrument of FIG. 14.

### DETAILED DESCRIPTION

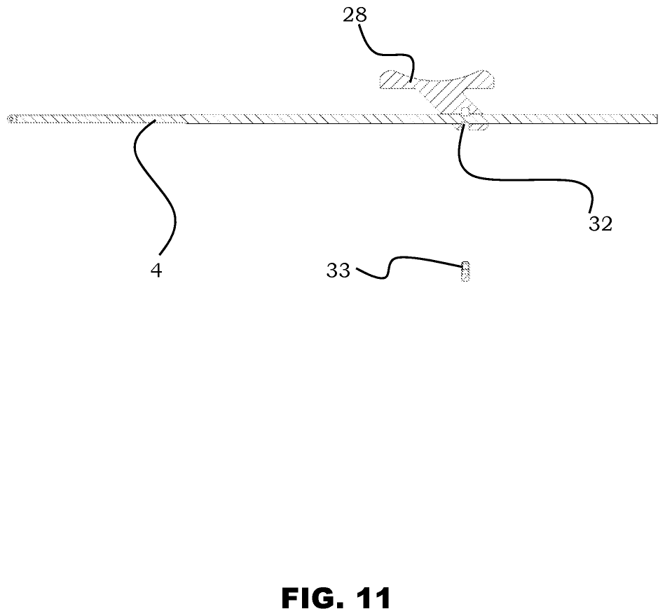

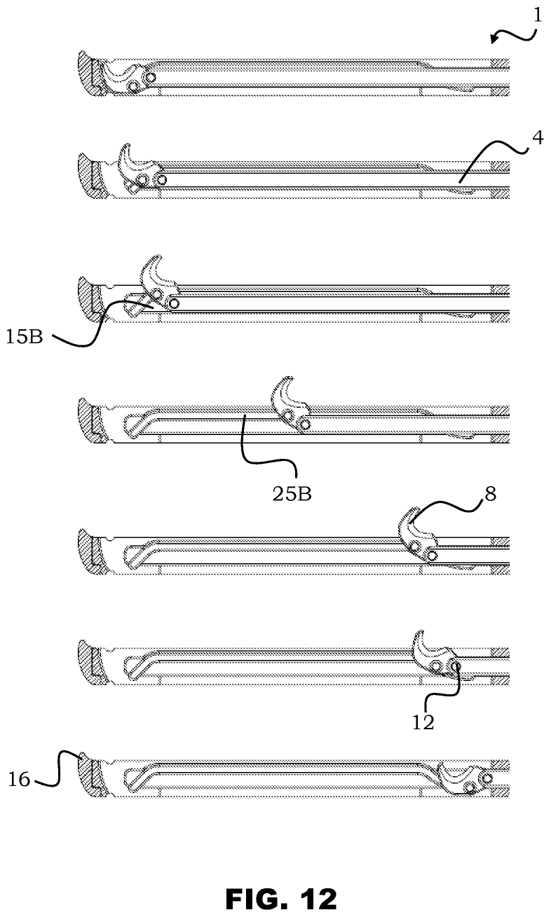

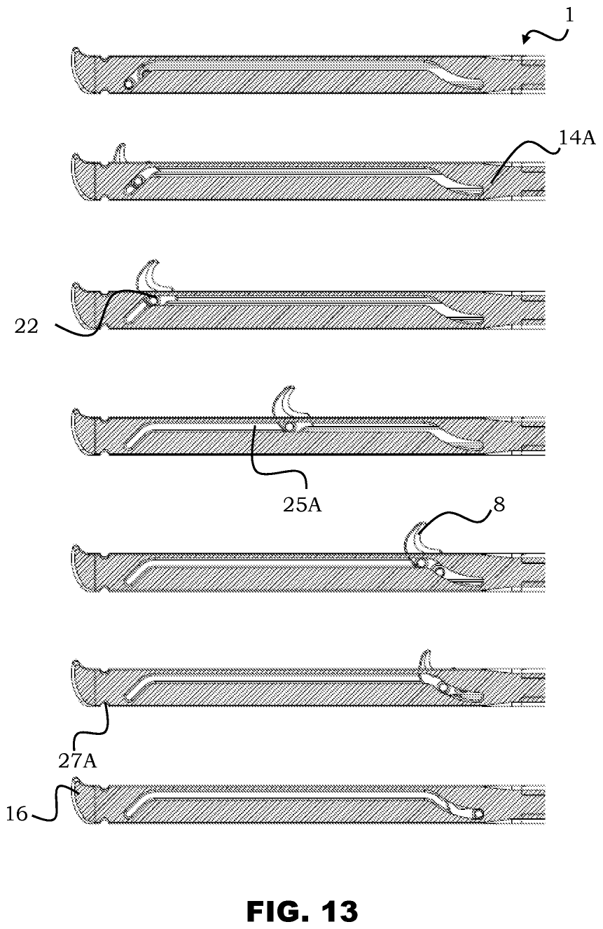

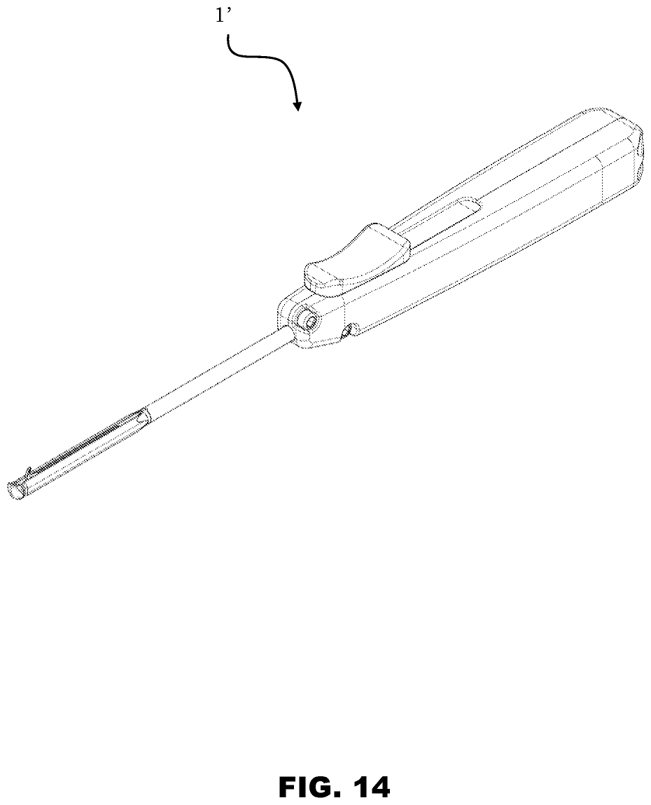

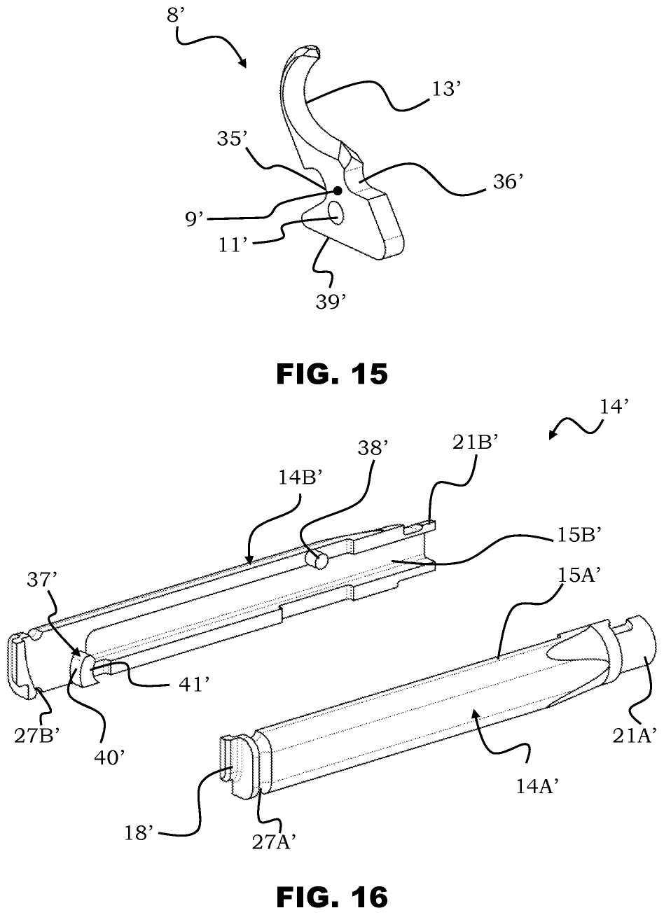

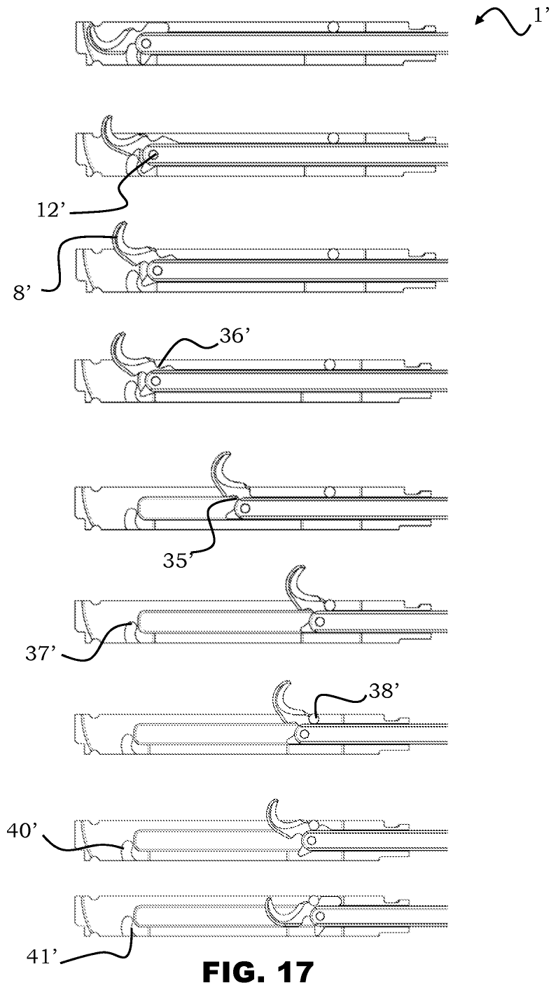

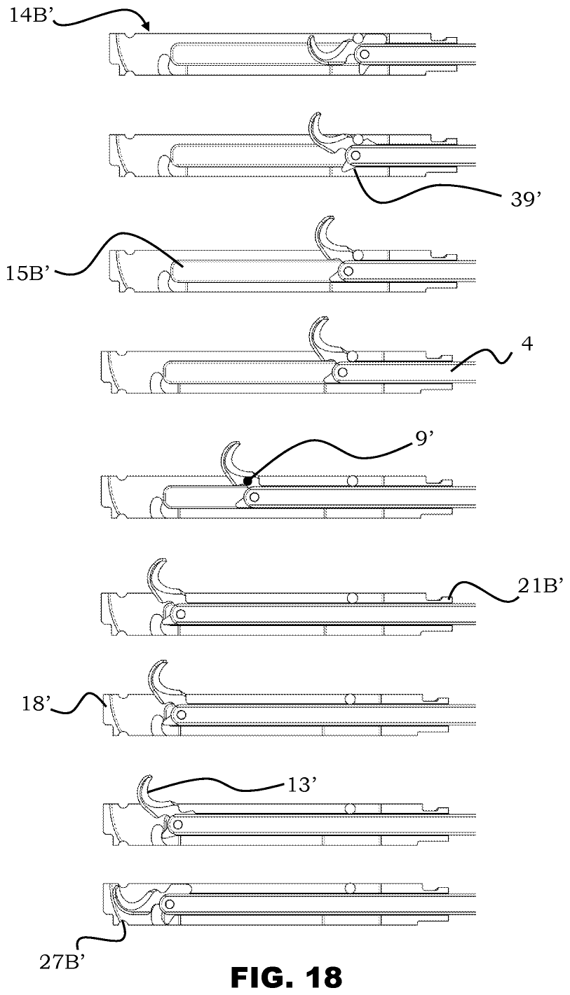

With reference to the enclosed FIGS. 1-13, reference number 1 globally and schematically indicates a first embodiment of a surgical cutting instrument made according to the present invention.

Specifically, the instrument 1 comprises a handle or gripping element 2. In the present embodiment this gripping element 2 is represented by a handle that can be grasped with just one hand by a surgeon in order to use the instrument 1.

A hollow stem 3, protruding from said gripping element 2 along a longitudinal axis L, is mounted on said gripping element 2.

In the present embodiment the gripping element 2 comprises a left gripping half shell 2A and a right gripping half shell 2B coupled to each other so as to enclose an end portion of the hollow stem 3.

Specifically, in said embodiment the assembly and constraint between left gripping half shell 2A and right gripping half shell 2B is made by means of screws 2C.

The left gripping half shell 2A is a mirror copy of the right gripping half shell 2B except for the screws 2C used and for an anti-rotation notch 2D that is present in just one of the two components and whose use will become clearer hereinafter.

The left gripping half shell 2A and the right gripping half shell 2B are shaped so as to keep the hollow stem 3 in a projecting position along the L axis.

In the hollow stem 3 a tie rod 4 is inserted or fitted through a proximal end 5 thereof. The rod 4 crosses the stem 3 until it penetrates into the gripping element 2 so that the proximal end 5 thereof is coupled to a sliding control lever 28 housed in the gripping element.

Opposite said proximal end 5, the tie rod 4 provides for a coupling portion 6 at a distal end 7.

A blade element 8, which is tooth-shaped, is rotatably mounted about a hinge positioned at the coupling portion 6 according to a transversal pitch axis with respect to the longitudinal development axis L of the instrument 1.

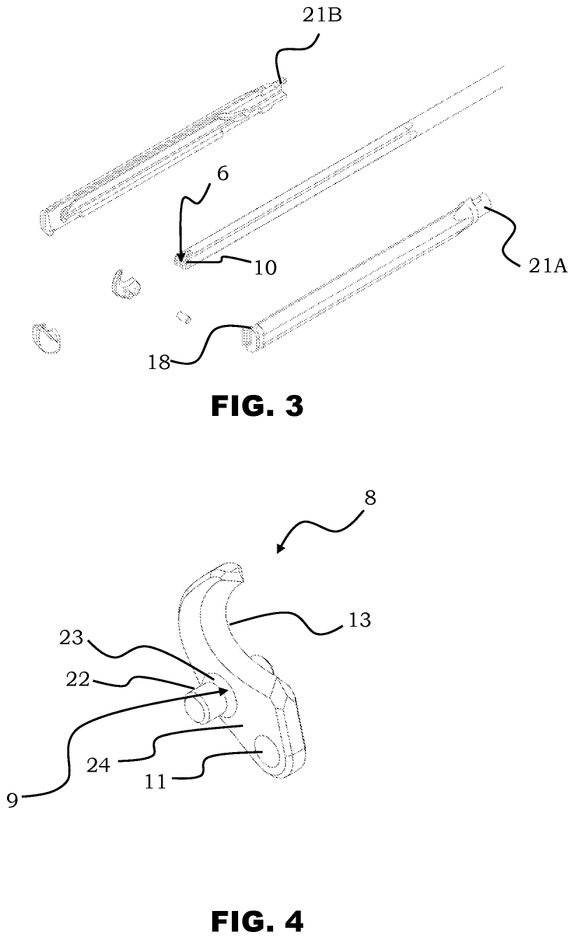

More particularly, as more visible in FIG. 3, the coupling portion 6 of the tie rod 4 is U-shaped, as an arch ring, and comprises a pair of engage holes 10 facing each other. The blade element 8 comprises a proximal hole 11, intended to be aligned coaxially with the engage holes 10 in order to receive a first connecting pin 12 adapted to be inserted into the engage holes 10 passing from the proximal hole 11 so as to make the hinge connection between tie rod 4 and blade element 8.

In the present exemplifying but not limiting embodiment represented in FIG. 4, the blade element 8 is substantially C-shaped, with a C-shaped inner cutting edge 13 and a fulcrum 9. In an alternative preferred embodiment the cutting edge 13 is shaped according to an angle of 135°, thus cutting as a knife.

The fulcrum 9 is the material point representing the movement of the blade element 8, the rotation along an arch of said fulcrum 9 bringing the blade element 8 from an inactive position in which it is hidden housed in the covering sheath and is parallel to the longitudinal development L of the cutting instrument 1 to an active position in order to carry out the cutting and transversal with respect to the longitudinal development L of the cutting instrument 1.

In the present embodiment, at the fulcrum 9 there is provided at least one pin 22 protruding parallel to the pitch axis and located in an almost central position under the profile of the cutting edge 13. More particularly, the pin 22 is provided on both sides of the blade element 8 protruding from opposite side surfaces 24 of the blade element 8 to allow better guide and stability of said element.

The blade element 8 further comprises a reinforcement base edge 23 for the pin 22 from which the pin itself protrudes at both side surfaces 24 of the blade element 8.

More particularly, in the present non-limiting embodiment, the pin 22 is formed integrally and embedded in the blade element 8 along with the base edge.

As it will be seen hereinafter in more detail, the blade element 8 is pulled by the rod 4 and can rotate about the hinge formed by the connecting pin 11 but, as stated, it also provides for the pin 22 sliding in a guide, the combination of hinge and pin 22 allowing for a controlled rotation of the fulcrum 9. Furthermore, the blade element 4 has a resistant end that takes up the form of the cutting edge 13 profile, in order to form a first-degree lever.

The cutting edge 13 of the blade element 8 has a sectional profile of less than 90° but nothing prevents from providing blades with different angles of the cutting edge 13, even greater than 90°.

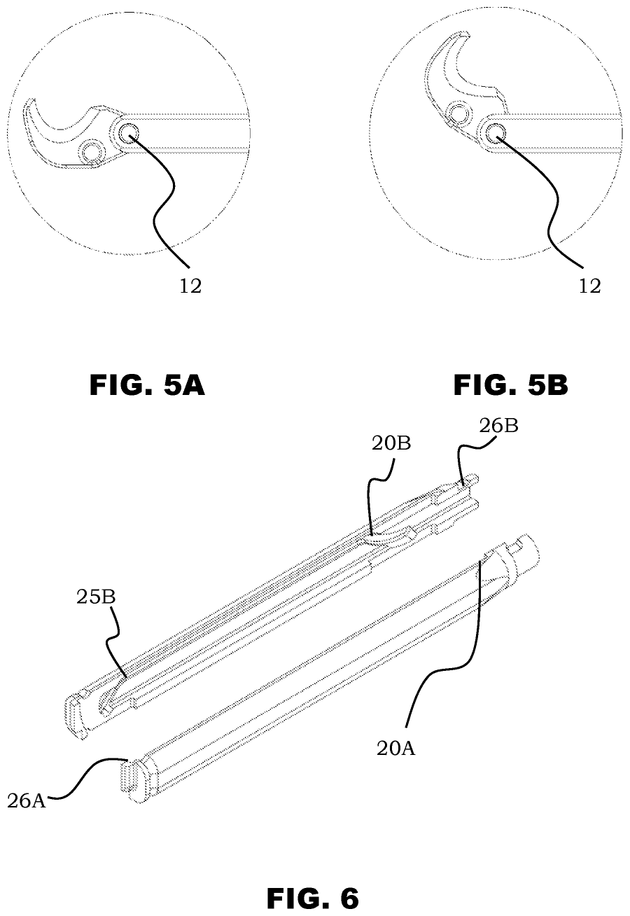

The blade element 8 may pass from an inactive or rest position, outlined and visible in FIG. 5A, to an active position, outlined and visible in FIG. 5B, and vice versa.

A sheath 14 or covering and guiding case 14 is further provided about an end portion of the tie rod 4 and the blade element 8.

In the inactive position, the blade element 8 is hidden housed inside the covering sheath 14; whereas in an active position the blade element 8 protrudes outwardly from the covering sheath 14.

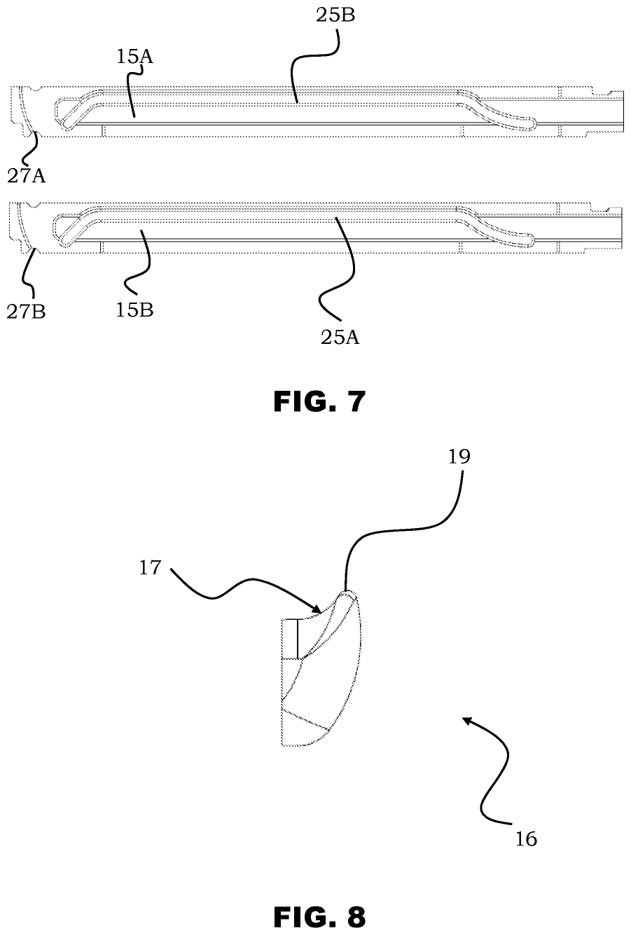

In the embodiment represented in examples of FIGS. 6 and 7, the covering sheath 14 comprises two facing portions with a left covering half shell 14A and a right covering half shell 14B coupled to each other to enclose, as stated, both the end portion of the tie rod 4, and the blade element 8.

In each half shell 14A, 14B, in the embodiment of the same Figures, a central slot or groove is present, 15A and 15B respectively, which mirror each other and have the purpose of keeping the tie rod 4 in the guide. When the two half shells are overlapped, the two facing grooves form a central duct in which the distal end of the tie rod 4 may slide.

Furthermore, in the present embodiment a tip 16, represented in FIG. 8, coupled to the instrument 1 at the blade element 8 and comprising a protuberance 17 for the detachment of the soft tissues is provided.

Moreover, said protuberance 17 provides a spatial positioning feedback of the instrument 1 in the working condition, for instance once it has been inserted into the palm arch.

Both the left covering half shell 14A and the right covering half shell 14B, at the own distal end facing toward the tip 16, comprise connecting elements 18 with anti-rotation elements, specifically anti-rotation planes 26A, intended to couple to the tip 16.

The retention seat of the tip 16 is shaped in order to house the connecting elements 18 with the anti-rotation planes 26A.

Once the tip 16 has been assembled to the anti-rotation planes 26A, the tip 16 itself will further act as distal retention element of the left covering half shell 14A and of the right covering half shell 14B in order to forma a unique body. From the opposite or proximal side of the sheath 14 anti-rotation coupling elements 21A and 21B are provided, which are intended to the fitting and retention function on the free end of the hollow stem 3.

The connecting and anti-rotation elements 18 ensure the alignment of the point 19 of the tip 16 with a longitudinal opening 20A and 20B present both in the left covering half shell 14A and in the right covering half shell 14B.

When the two half shells are coupled to each other, the two openings 20A and 20B form a unique longitudinal window through which the point of the cutting edge 13 comes out when the blade element 8 is made operational.

Furthermore, at the connecting elements 18, and more specifically on a surface opposite the one of the blade-element 8 exit window, reference recesses 27A and 27B are provided, which are useful to signal the exit point of the blade element 8, being detectable by the ultrasounds, thus generating a discontinuity of the surface and therefore a different wave refraction.

In other words, they are used by the surgeon to understand the position in which the end of the instrument 1 is located before performing a cut.

The central slot 15A and 15B for each half shell 14A, 14B is closed from the side of the tip 16 whereas it is open at the opposite end, namely from the side of the hollow stem 3.

At the hollow stem 3, the ends 21A and 21B of the left covering half shell 14A and of the right covering half shell 14B are half cylinders with a further anti-rotation element, an anti-rotation notch 26B, and are designed to be assembled with the hollow stem 3. Said ends 21A and 21B are also useful to ensure the alignment of the hollow stem 3 with a window formed by the two openings 20A and 20B present in the left covering half shell 14A and in the right covering half shell 14B, as well as for the alignment of the tip 16.

In the exemplifying embodiment of FIGS. 1 to 13, as stated, the blade element 8 provides for the pin 22, at the fulcrum 9, to protrude from both parts.

Both the left covering half shell 14A and the right covering half shell 14B of the covering sheath 14 comprise each an inner guide groove 25A, 25B.

In the inner guide grooves 25A, 25B the pin 22 of each side of the blade element slides, actually making the fulcrum 9 of said blade element 8 movable.

In the present embodiment the inner guide grooves 25A, 25B are configured with three contiguous segments, of which: two tilted and one parallel with respect to the central groove.

In the tilted section at the tip 16 the sliding of the pin 22 of the blade element 8 allows the blade to exit, in the plane section the blade, in the extracted position, cuts the tissues in contact therewith, whereas in the last tilted section the blade element 8 returns to the safety covered position in order to allow the extraction of the instrument 1.

In other words, the pin 22 is adapted to slide along the inner guide groove formed by the coupling of the grooves 25A, 25B to allow the blade element 8 to exit and be hide-retained during a sliding or retraction of the tie rod 4.

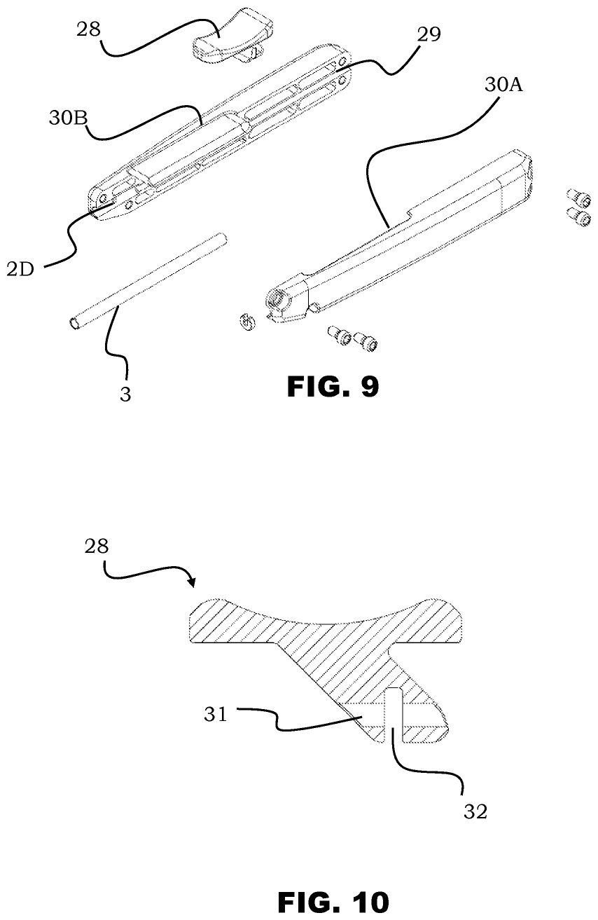

As more visible in FIGS. 9 to 11 at the gripping element 2 a control lever 28 command is provided, which is operatively connected to the tie rod 4 for a longitudinal movement thereof along with the blade element 8.

The anti-rotation notch 2D ensures the alignment of the point of the tip 16 and the upper opening 20A, 20B with the gripping element 2 in the direction where the control lever 28 is present.

Moreover, the notch 2D prevents the hollow stem 3 from decoupling from the gripping element 2.

A hole 29 is provided, which allows the tie rod 4 to pass through the gripping element 2 at the moment the control lever 28 is pulled.

The left covering half shell 14A and the right covering half shell 14B provide each for respective grooves 30A, 30B which keep the control lever 28 in position and guide the sliding thereof into the gripping element 2.

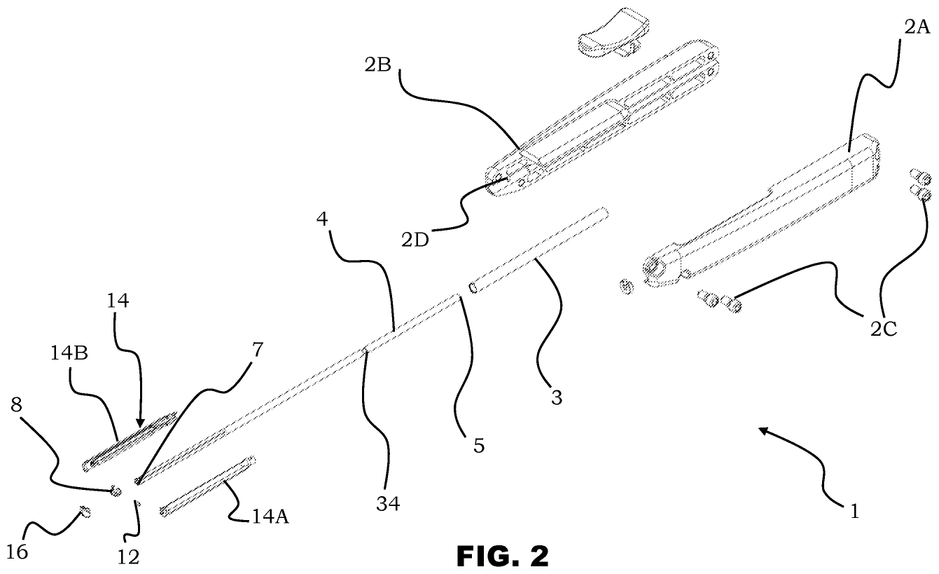

The control lever 28 couples to the tie rod 4 through a seat 31 formed in the gripping element 2. A cavity 32 operatively associated with a locking ring 33 allows making said components further integral by a recess 34, visible in FIG. 2.

In FIGS. 12 and 13 a work sequence of the blade element 8 is summed up in an intervention at the carpal tunnel when the control lever 28 is operated according to the embodiment described above.

In FIG. 12 a longitudinal section is represented, which allows appreciating the movement of the blade element 8 in its entirety whereas in FIG. 13 a section is represented, which allows appreciating the movement of the second pin 22, with relative movement of the fulcrum 9, only in the guide groove 25A.

At first, the blade is completely hidden within the left covering half shell 14A and the right covering half shell 14B coupled to form the sheath 14.

In this way, the surgeon can insert the instrument 1 into the palmar arch in complete safety without risking to cut unwanted tissue.

The surgeon, indeed, operates in confined spaces and close to the median nerve and the ulnar artery.

Therefore, it is absolutely essential that no damage is done to these tissues and nerve endings.

When the instrument is correctly positioned inside the carpal canal, by operating the control lever 28 the movement is transmitted to the tie rod 4 which translates backwards in the proximal direction while remaining in the central position.

Conversely, the pin 22 slides in the inner guide grooves 25A, 25B of the left covering half shell 14A and of the right covering half shell 14B. This results in the movement of the fulcrum 9 and in a rotation of the blade element 8 along the three segments of the guide groove which comprises two tilted sections and a plane section.

Therefore, the blade element 8 rises to move into the active position by exiting from the sheath's window. From now on the blade slides horizontally in order to resect the transverse carpal ligament.

Afterwards, by continuing the sliding, the blade element returns into the covering sheath 14 allowing the surgeon to extract the instrument 1 from the carpal canal in complete safety.

In FIGS. 14 to 18 a second alternative embodiment of a surgical cutting instrument 1′ according to the present invention is represented.

The substantial differences compared to the first embodiment lie in the shape of a blade element 8′ and in the one of a covering sheath 14′.

The same elements are herein indicated with the same reference numbers as of the previous embodiment.

The blade element 8′ in this case as well, but just by way of non-limiting example, is C-shaped with a fulcrum 9′, and a first pin 12′, below a cutting edge 13′ of the blade element 8′. In a preferred alternative embodiment the cutting edge 13′ is shaped according to an angle of 135°, thus cutting as a knife.

The fulcrum 9′ is once again a material point representative of the movement of the blade element 8′.

In this case, as it will be clearer hereinafter, at said fulcrum 9′ no pin is provided.

The blade element 8′ comprises a race portion 35′ below the surface of the blade element 8′ opposite the cutting edge 13′.

In the present embodiment a second race portion 36′ is further provided below and adjacent the cutting edge 13′.

The covering sheath 14′ comprises at least one guide element 37′ adapted to be coupled with the race portion 35′ to allow a movement of the tie rod 4 and of the blade element 8′.

In the present embodiment a second guide element 38′ is further provided.

In the present embodiment the guide element 37′ is represented by an arch protuberance, with the concavity facing the gripping element 2, made close to the distal element 16 and extending from a lower surface of the instrument 1′ up to about a midline of the instrument 1′.

The second guide element 38′ is instead shaped as a cylindrical pin made close to the gripping element 2 at an upper surface of the instrument 1′.

Nothing prevents from adopting different shapes for the guide element 37′ and for the second guide element 38′.

The race portion 35′ guides the movement of the blade element 8′ in the distal part of the covering sheath 14′ and is substantially U-shaped with the upper portion adapted to guide the movement to make the blade element 8′ exit, and the lower portion adapted to guide the movement to make the blade element 8′ enter again.

The second race portion 36′ instead guides the movement of the blade element 8′ close to the gripping element 2.

It is also substantially U-shaped with the upper portion adapted to guide the movement to make the blade element 8′ enter again, and the lower portion adapted to guide the movement to make the blade element 8′ exit again.

Therefore, in this embodiment it is the combined action of the hinge, the race portion 35′, the second race portion 36′, the guide element 37′, the second guide element 38′, and the base 39′ when it comes into contact with the guide 15 that allows the movement in controlled rotation of the fulcrum 9′.

In other words, it is as if the blade element 8′ was mounted on a pair of hinges, wherein the first hinge is represented by the first pin 12′ and actually is a carriage-mounted hinge since it moves inside the covering sheath 14′ being hooked to the end of the tie rod; the second hinge is represented by the movable fulcrum 9′ which, in the case of this second alternative embodiment, is configured by the cooperation of the profiles of the race portion 35′ and of the second race portion 36′ with the guide element 37′ and with the second guide element 38′.

The base 39′ of the blade element 8′ guides and stabilizes the position during the cutting phase, but also during the return phase in case it is necessary to perform a second passage to complete the cut.

The covering sheath 14′ is made, analogously to the first embodiment, by a left covering half shell 14A′ and a right covering half shell 14B′, substantially mirrored to each other, except for the guide element 37′ and the second guide element 38′ provided only on one of the two.

In the present embodiment as well, the left covering half shell 14A′ and the right covering half shell 14B′ comprise a central slot 15A′ and 15B′ which guides the blade element 8′.

Connecting elements 18′ are also provided, which connect the tip 16 and keep the left covering half shell 14A′ and the right covering half shell 14B′ joined together.

At the shaft element 3, the ends 21A′ and 21B′ of the left covering half shell 14A′ and right covering half shell 14B′ are half cylinders with an anti-rotation notch 26B′ and are intended to be assembled with the shaft element 3′, as well as to ensure the alignment of the shaft element 3 with an upper opening 20A′ and 20B′ present in the left covering half shell 14A′ and right covering half shell 14B′ and for aligning the tip 16.

Furthermore, at the connecting elements 18′, and more specifically on a surface opposite the extraction one of the blade element 8′ reference recesses 27A′ and 27B′ are provided, which are useful for signaling the exit point of the blade element 8′ from the moment they are visible by ultrasounds, thus generating a discontinuity of the surface and thus a different wave refraction.

They are therefore useful for the surgeon to understand the position of the instrument 1′ before performing the cut.

In the present embodiment the guide element 37′ at a first surface 40′ opposite the concavity guides the movement to make the blade element 8′ exit, whereas at a second surface 41′ it guides the movement to make the blade element 8′ enter again.

The second guide element 38′ guides the movement of the blade element 8′ in the proximal part of the instrument 1′, at the gripping element 2.

The arch of the cylinder facing toward the tip 16 guides the movement to make the blade element 8′ enter again, whereas the arch of the cylinder facing towards the gripping element 2 guides the movement to make the blade element 8′ exit.

In FIG. 17 a work sequence of the blade element 8′ is summed up in an intervention at the carpal tunnel when the sliding lever 28 is operated according to this second embodiment described above.

In the phase when the blade element 8′ comes out, the tie rod 4 drags the blade element 8′ and consequently the fulcrum 9′ towards the gripping element 2.

The base 39′ comes into contact with the guide 15 causing it to rotate. The upper portion of the race portion 35′ comes into contact with the first surface 40′ of the guide element 37′ causing the rotation of the blade element 8′.

This phase ends when the contact ends and the base 39′ starts to be in contact and to slide in the central slot 15A′, 15B′ of the left covering half shell 14A′ and right covering half shell 14B′.

In the cutting phase, the base 39′ is in contact with the central slot 15A′, 15B′ which blocks the rotation thereof and stabilizes the blade element 8′.

In an alternative embodiment not shown, the base 39′ may be made of two segments.

In the return or recovery phase of the blade element 8′, the upper portion of the second race portion 36′ comes into contact with the second guide element 38′ which causes the rotation of the blade element 8′ and the movement of the fulcrum 9′ the moment the tie rod 4 exerts its pulling force and drags the system towards the gripping element 2. The movement ends with the contact of the blade element 8′ with the central slot 15A′, 15B′.

In this position the instrument 1′ may be easily extracts by the surgeon without running the risk of damaging the surrounding tissues since the blade is completely contained inside the instrument 1′.

In other words, the present embodiment is structurally mirrored to the first embodiment, with the protruding elements placed on the covering sheath 14′ and the mutual grooves placed on the blade element 8′ differently from the first case in which the protruding element, namely the pin 22, is placed on the blade element 8, whereas the inner guide grooves 25A, 25B are provided in the covering sheath 14. However, the effect of inducing the rotation of the latter through mutual abutment by means of the relative movement controlled by the user is analogous.

As visible specifically in FIG. 18, it is optionally possible to perform a phase of re-entry into the distal position, which is only necessary in the event that it is necessary to repeat a second passage to complete the ligament cutting.

In this case the blade element 8′ must return to its initial position.

By pushing the tie rod 4 in the opposite direction, the lower portion of the second race portion 36′ comes into contact with the second guide element 38′ which causes the blade element 8′ to rotate.

The movement ends with the contact of the base 39′ with the central slot 15A′, 15B′.

The lower portion of the second race portion 36′ remains in contact with the second guide element 38′, thus avoiding a blockage of movement.

Once the blade element 8′ ends the stroke along the central slot 15A′, 15B′, the lower portion of the race portion 35′ comes into contact with the surface 41′ causing the rotation of the blade element 8′ and the movement of the fulcrum 9′ when it is pushed by the tie rod 4.

The phase ends when the blade element 8′ is completely contained within the covering sheath 14′.

Advantageously it is possible for a surgeon to operate in total safety even in the presence of multiple close-up tissues while maintaining a full haptic perception of the tissues touched.

The instrument of the present invention is simple to be made from a production point of view and therefore suitable for large-scale production and distribution.

Finally, the instrument according to the invention can be made with materials suitable for surgical instruments and capable of being sterilized and reused according to industry standards.

The person skilled in the art will understand that the embodiment presented may be subjected to further changes and variations, according to specific and contingent needs, all included within the scope of protection of the invention as defined by the following claims.

Indeed, nothing prevents from providing variations in the conformation of components such as blade element or covering sheath according to different needs, while remaining within the scope of protection defined by said claims.

1. Surgical cutting instrument for minimally invasive surgery comprising:
a gripping element;
a hollow stem mounted as an extension of said gripping element along a longitudinal axis of the instrument;
a tie rod housed in the hollow stem and having a proximal end coupled to a control lever in said gripping element and a distal end hinged to a blade element; and
a covering and guiding sheath for said blade element as an extension of said hollow stem;
said blade element having a movable fulcrum in said covering and guiding sheath and being configured to pass through an inactive position in which it is hidden housed in said covering sheath and an active position protruding outwardly through a window of said covering sheath and vice versa.

2. Surgical cutting instrument according to claim 1, wherein said distal end of said tie rod is U-shaped, as an arch ring, and comprises a pair of engage holes facing each other and wherein the blade element comprises a proximal hole coaxially aligned with the engage holes to receive a first pin so as to make the hinging connection between tie rod and the blade element.

3. Surgical cutting instrument according to claim 2, wherein said blade element comprises a second pin at said fulcrum and said covering sheath comprises at least one inner guide groove, said second pin being adapted to be movably guided by said tie rod in said at least one inner guide groove making said fulcrum movable.

4. Surgical cutting instrument according to claim 1, wherein said blade element comprises at least one race element and said covering sheath comprises at least one guide element, said at least one race element is adapted to operationally abut with said at least one guide element by a movement of said tie rod, making said fulcrum movable.

5. Surgical cutting instrument according to claim 1, wherein the covering sheath comprises two portions facing each other with a left covering half shell and a right covering half shell coupled to each other to enclose both the end portion of the tie rod, and the blade element.

6. Surgical cutting instrument according to claim 1, further comprising a tip coupled at said blade element and comprising a protuberance for the detachment of the soft tissues, said protuberance being further adapted to provide a spatial positioning feedback of said surgical cutting instrument in working condition.

7. Surgical cutting instrument according to claim 1, wherein said control lever is housed and sliding in a corresponding seat formed in said gripping element.

8. Surgical cutting instrument according to claim 1, wherein said gripping element comprises a left gripping half shell and a right gripping half shell coupled to each other so as to enclose a portion of said hollow stem.

9. Surgical cutting instrument according to claim 1, wherein said blade element is C-shaped.

10. Surgical cutting instrument according to claim 1, further comprising reference recesses which can be detected by ultrasounds to identify a position of said instrument with respect to surrounding tissues.
